(19) United States
(12) Patent Application Publication
(10) Pub. No.: US 2022/0202460 A1
(43) Pub. Date: Jun. 30, 2022

(54) REVERSE THREAD BONE SCREW

(71) Applicant: University of Maryland Medical Center, LLC, Baltimore, MD (US)

(72) Inventor: Natalie Chilaka, Baltimore, MD (US)

(21) Appl. No.: 17/563,405

(22) Filed: Dec. 28, 2021

Related U.S. Application Data
(60) Provisional application No. 63/131,313, filed on Dec. 28, 2020.

(51) Int. Cl. A61B 17/86

(52) U.S. Cl. CPC A61B 17/8625

(57) ABSTRACT

Reverse thread bone screws and more particularly reverse thread bone screws with a helix angle that slopes upward to the left such that counter clockwise rotation drives forward axial advancement of the screw.

### PRIORITY INFORMATION

This application is a non-provisional application that claims the benefit of U.S. Provisional Application Ser. No. 63/131,313, filed on Dec. 28, 2020, the contents of which are herein incorporated by reference in their entirety.

### TECHNICAL AREA

The embodiments of the present invention generally relate to reverse thread bone screws, and more particularly, toward reverse thread bone screws with a helix angle that slopes upward to the left such that counter clockwise rotation drives forward axial advancement of the screw.

### BACKGROUND OF THE INVENTION

There are certain anatomic locations and fracture patterns where the clockwise rotational forces produced during traditional thread screw insertion can compromise fracture reduction.

Rotational forces produced during screw insertion can be particularly problematic when placing lag screws—including both lag by technique with over-drilling of the near fragment and use of a fully thread screw or lag by design with a partially threaded screw. When inserting a lag screw there is no thread purchase in the near fragment, so when the screw thread engages the far fragment a rotational force differential is concentrated at the fracture site. This concentrated force can be powerful enough to produce loss of fracture reduction with rotational deformity through the fracture, even when the trajectory of the lag screw is perfectly perpendicular to the plane of the fracture.

Typical human anatomy has 15-20° of femoral neck anteversion. When fixing left sided femoral neck, basicervical, intertrochanteric and some subtrochanteric hip fractures, the clockwise rotational forces during traditional thread lag screw insertion can cause an extension deformity through the fracture. This can result in poor fracture reduction with increased risk of construct failure, sometimes necessitating additional surgery, and compromised clinical outcomes. When fixing right sided hip fractures, the same clockwise rotational force during traditional thread lag screw insertion can impart a flexion force across the fracture. This flexion force typically does not result in a detrimental flexion deformity when fixing right sided hip fractures due to femoral neck anteversion where displacement of the proximal head fragment into flexion is resisted by contact with, and even compression against, more posterior bone.

Alternate techniques for counteracting the clockwise rotational forces during traditional thread lag screw insertion for fixation of left sided hip fractures include:
a. Use of a separate “derotational screw”: Placement of a smaller diameter screw across the fracture prior to placement of the primary hip lag screw to help resist rotational forces generated during lag screw insertion. This approach can be used with sliding hip screw constructs but not cephalomedullary nail constructs as the proximal portion of the nail blocks the trajectory for a derotational screw. Use of a derotational screw with a sliding hip screw construct increases the risk of an iatrogenic lateral femoral cortex fracture, which would necessitate addition of a trochanteric stabilizing plate to the sliding hip screw construct or even conversion to a cephalomedullary nail construct.
b. Placement of additional k-wires across the fracture prior to lag screw insertion to increase provisional fracture stability and resist rotational forces produced during lag screw insertion. The approach is technically more challenging with cephalomedullary nail constructs but can be performed successfully with both sliding hip screw constructs and cephalomedullary nail constructs.
c. Use of a bone hook, ball spike or larger diameter k-wire or Schanz pin to manipulate the proximal segment of the fracture and resist rotational forces produced during lag screw insertion.
d. Placement of a clamp across the fracture to stabilize the fracture reduction resist rotational forces produced during lag screw insertion. This is more difficult with percutaneous techniques commonly used in surgical fixation of hip fractures.
e. Tapping for the lag screw and/or multiply inserting and removing the lag screw while using additional techniques to control rotational forces described above. This will decrease the purchase of the lag screw in the bone and so lessen the deforming rotational forces generated during lag screw insertion; however, this also sacrifices quality of implant fixation.
f. Use of a supplemental plate to further stabilize the fracture prior to lag screw insertion. This technique may be employed during an open approach to a femoral neck fracture, such as a high energy high angle femoral neck fracture in a younger individual, where the plate may also serve a buttress function to resist sheer forces that also act on this fracture pattern and contribute to construct failure, and so may be employed in the surgical treatment of both left and right sided hip fractures.

### SUMMARY OF THE INVENTION

Accordingly, the embodiments of the present invention are directed to reverse thread bone screws that substantially obviate one or more problems due to limitations and disadvantages of the related art.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the reverse thread bone screw includes a substantially cylindrical body portion having a longitudinal axis with reverse angle threads formed around a distal end of the body portion, and a unique drive coupling formed in a proximal end of the body portion, and a predefined radiographic identifier adhered to or embedded in the body portion.

In another aspect, the reverse thread bone screw includes a reverse thread hip lag screw comprising a substantially cylindrical body portion having a longitudinal axis with reverse angle threads formed around a distal end of the body portion, and a drive coupling formed in a proximal end of the body portion, and a predefined radiographic identifier adhered to or embedded in the body portion.

In another aspect, the reverse thread screw comprises a substantially cylindrical body portion having a longitudinal axis with reverse angle threads formed around a distal end of the body portion and the reverse angle threads extending the length of the body portion to terminate adjacent to a proximal end of the body portion, and a drive coupling formed in the proximal end of the body portion, and a predefined radiographic identifier adhered to or embedded in the body portion.

In another aspect, a reverse thread screw kit comprises a reverse thread screw comprising a substantially cylindrical body portion having a longitudinal axis with reverse angle threads formed around a distal end of the body portion and the reverse angle threads extending the length of the body portion to terminate adjacent to a proximal end of the body portion, and a drive coupling formed in the proximal end of the body portion, and a predefined radiographic identifier adhered to or embedded in the body portion, a reverse thread tap configured to provide reciprocally configured threads to engage the threads of the reverse thread screw, and a reverse thread drive mechanism reciprocally configured to engage the drive coupling of the reverse thread screw.

It is to be understood that both the foregoing general description and the following detailed description are exemplary and explanatory and are intended to provide further explanation of the invention as claimed.

### BRIEF DESCPRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 is a side view of a related art thread screw.

FIG. 2 is a side view of a related art thread hip lag screw.

FIG. 3 is a side view of a reverse thread screw, in accordance with an embodiment of the disclosed subject matter.

FIG. 4 is a side view of a reverse thread hip lag screw, in accordance with an embodiment of the disclosed subject matter.

FIG. 5 is a plan view of an end of a head of a reverse thread-type screw with a uniquely-configured drive coupling, in accordance with an embodiment of the disclosed subject matter.

FIG. 6 is a plan view of an end of a head of a reverse thread-type screw with a uniquely-configured drive coupling, in accordance with another embodiment of the disclosed subject matter.

### DETAILED DESCPRIPTION

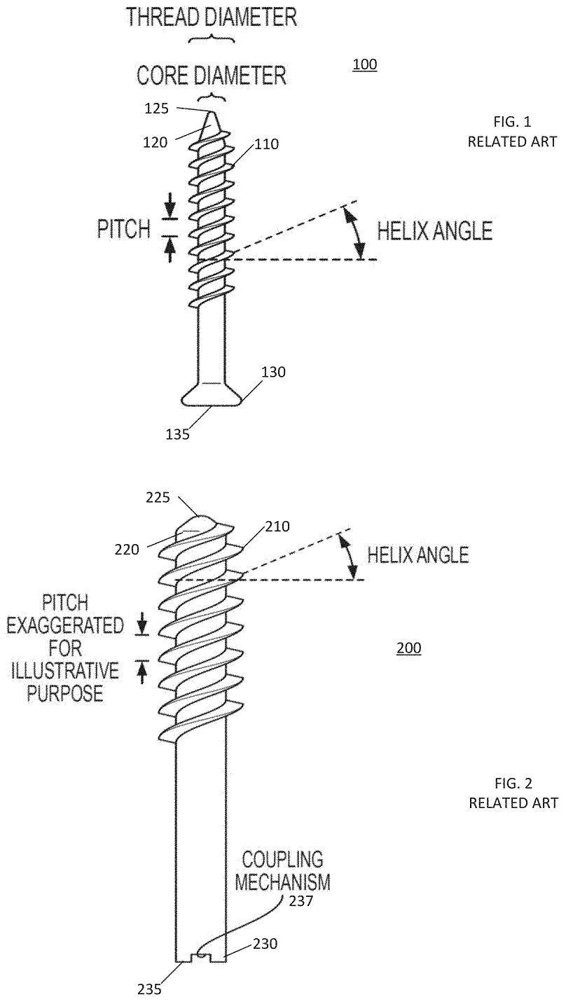

In general, traditionally threaded bone screws have a helix angle that slopes upward to the right such that clockwise rotation drives forward axial advancement of the screw. However, the embodiments of the present invention provide reverse thread bone screws having a helix angle that slopes upward to the left such that counter clockwise rotation would drive forward axial advancement of the screw. Screw dimensions and thread pitch may be the same or similar as the traditional thread bone screw counterpart, as shown in FIGS. 1-4.

Implementation of a reverse thread hip lag screw for fixation of appropriate left sided hip fractures is a novel approach that would convert extension deforming causing clockwise rotational forces produced during traditional thread lag screw insertion into more favorable counterclockwise rotational forces that are better resisted by normal hip anatomy and better tolerated. Production of a counterclockwise rotational force during lag screw insertion for the fixation of left sided hip fractures would obviate the need for using the additional maneuvers described for many, if not most, of these fractures. Not needing to employ additional techniques to maintain fracture reduction may further benefit patient care and cost of treatment by decreasing operative time and potentially decreasing the number of implants.

A reverse thread bone screw should have a unique radiographic identifier such that a reverse thread bone screw can be identified in situ by x-ray alone:
a. Typical thread pitch of hip lag screws is small enough that x-rays would have to be carefully scrutinized to distinguish traditional thread and reverse thread hip lag screws in situ.
b. Protect from interpretation error resulting from inadvertent reversal or mislabeling of an image.
c. Of particular importance if implant removal is required so that the screw is not inadvertently advanced instead of extracted, which could result in serious iatrogenic injury, i.e., doctor caused injury.
d. A unique radiographic identifier could take several forms, such as but not limited to a carbide ring at the base of the threads or bead(s) of increased radiographic density.

Additional instrumentation to accompany reverse thread bone screws:
e. A reverse thread tap should accompany reverse thread bone screws where the helix angle slopes upward to the left such that counter clockwise rotation would drive forward axial advancement and the dimensions and thread pitch of the reverse thread tap are the same as the corresponding traditional thread tap.
f. A unique couple mechanism or screw driver for reverse thread bone screw insertion and extraction may be used as an additional failsafe against a surgeon incorrectly advancing or extracting a screw when both traditional thread and reverse thread bone screws are available on the set.
g. No additional instrumentation or modifications would be necessary to employ reverse thread hip lag screws with most existing sliding hip screw and cephalomedullary nail systems. One exception is the Smith+Nephew Trigen Intertan Intertrochanteric Antegrade Nail system, which requires manufacture of a reverse thread compression screw for use with a reverse thread hip lag screw.

FIG. 1 is a side view of a related art thread screw. In FIG. 1, a traditional thread screw 100, that is, a regular or right-hand threaded screw 100 is shown with a thread 110 around an inner core 120 and the thread 110 having a helix angle a sloping upward from the left to the right. A diameter of the thread 110 is greater than a diameter of the inner core 120. The inner core 120 includes a conical front, distal end 125 and a head 130 proximal end opposite the conical from end. The head 130 also includes a drive mechanism in a proximal end 135 that is configured to engage a drive tool, for example, a screw driver. The clock-wise rotation of the traditional threaded screw 100 drives the axial movement of the traditional threaded screw 100 into an object. The thread 110 extends substantially the entire length of the inner core 120 from adjacent the conical front end 125 to adjacent a distal end of the head 130.

FIG. 2 is a side view of a related art thread hip lag screw. In FIG. 2, a traditional thread hip lag screw 200, that is, a regular or right-hand threaded hip lag screw 200 is shown with a thread 210 around an inner core 220 and the thread 210 having a helix angle a sloping upward from the left to the right. A diameter of the thread 210 is greater than a diameter of the inner core 220. The inner core 220 includes a conical front, distal end 225 and a head 230 proximal end opposite the conical from end. The head 230 also includes a drive mechanism 237 in a proximal end 235 that is configured to engage a drive tool, for example, a screw driver. The clock-wise rotation of the traditional threaded screw 200 drives the axial movement of the traditional threaded screw 200 into an object. The thread 210 extends only a portion of the length of the inner core 220 from adjacent the conical front end 225 to about ¼ to ½ the length of the inner core 220.

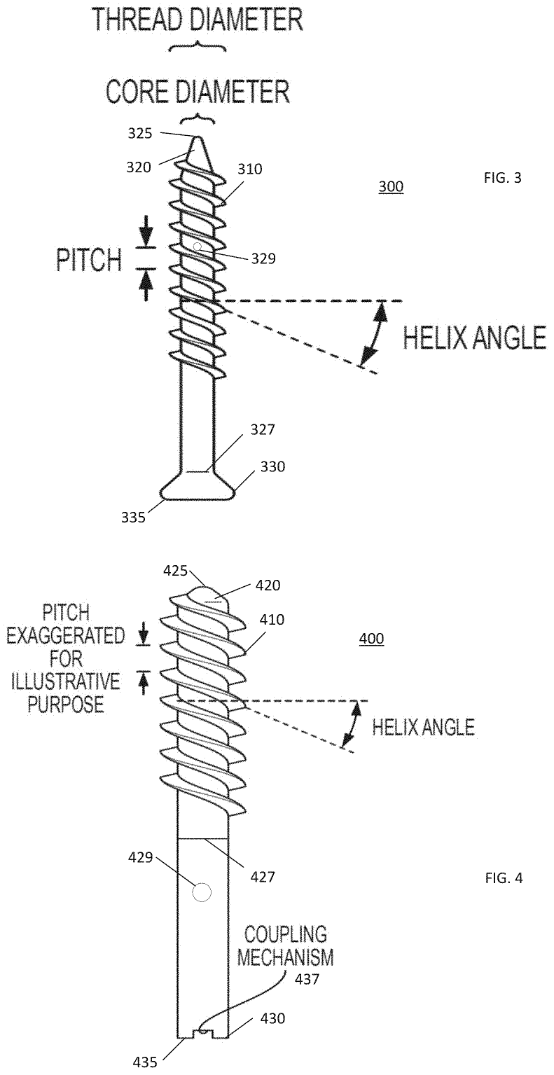

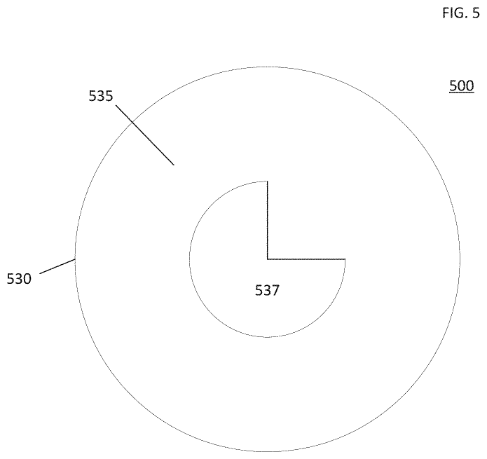

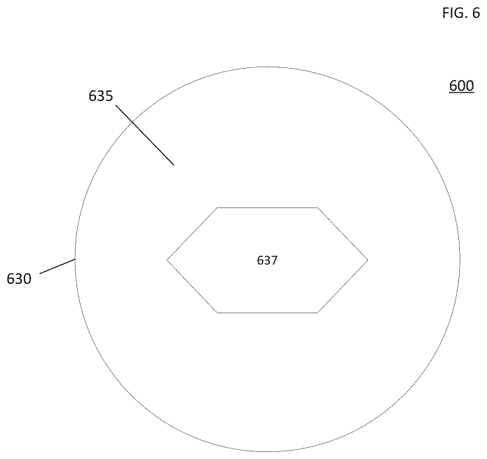

FIG. 3 is a side view of a reverse thread screw, in accordance with an embodiment of the disclosed subject matter. In FIG. 3, a reverse thread screw 300, that is, a reverse or left-hand threaded screw 300 is shown with a reverse thread 310 around an inner core 320 and the reverse thread 310 having a helix angle a sloping upward from the right to the left. A diameter of the reverse thread 310 is greater than a diameter of the inner core 320. The inner core 320 includes a conical front, distal end 325 and a head 330 proximal end opposite the conical from end. To aid in the identification of the type of screw before surgery, a radiologic marker, for example, but not limited to, one or more carbide bands 327 may be attached to the inner core 320, for example, but not limited to, adjacent to a base of the reverse thread 310 where the base is distal to the conical front, distal end 325. Alternatively, or in addition to the carbide band(s) 327, one or more radiologic beads 329 may be attached along the inner core 320. The head 330 also may include a uniquely-configured drive coupling (see FIGS. 5 and 6 for exemplary embodiments of the drive coupling) in a proximal end 335 of the reverse threaded screw 300 that is configured to engage a reciprocally-shaped drive tool, for example, a uniquely-shaped screw driver and/or other drive tool. The counter clock-wise rotation of the reverse threaded screw 300 drives the axial movement of the reverse threaded screw 300 into an object. The reverse thread 310 extends substantially the entire length of the inner core 320 from adjacent the conical front end 325 to adjacent a distal end of the head 330.

FIG. 4 is a side view of a reverse thread hip lag screw, in accordance with an embodiment of the disclosed subject matter. In FIG. 4, a reverse thread hip lag screw 400, that is, a reverse or left-hand threaded hip lag screw 400 is shown with a reverse thread 410 around an inner core 420 and the reverse thread 410 having a helix angle a sloping upward from the left to the right. A diameter of the reverse thread 410 is greater than a diameter of the inner core 420. The inner core 420 includes a conical front, distal end 425 and a head 430 proximal end opposite the conical from end. To aid in the identification of the type of screw before surgery, a radiologic marker, for example, but not limited to, one or more carbide bands 427 may be attached to the inner core 420, for example, but not limited to, adjacent to a base of the reverse thread 410 where the base is distal to the conical front, distal end 425. Alternatively, or in addition to the carbide band(s) 427, one or more radiologic beads 329 may be attached along the inner core 420. The head 430 also may include a uniquely-configured drive coupling (see FIGS. 5 and 6 for exemplary embodiments of the drive coupling) in a proximal end 435 reverse threaded screw 400 that is configured to engage a drive tool, for example, a screw driver and/or other drive tool. The counter clock-wise rotation of the reverse threaded screw 400 drives the axial movement of the traditional threaded screw 200 into an object. The reverse thread 410 extends only a portion of the length of the inner core 420 from adjacent the conical front end 425 to about ¼ to ½ the length of the inner core 420.

FIG. 5 is a plan view of an end of a head of a reverse thread-type screw with a uniquely-configured drive coupling, in accordance with an embodiment of the disclosed subject matter. In FIG. 5, a head 530 of a reverse thread-type screw 500, which can include, for example, but is not limited to, the reverse thread screw 300 of FIG. 3, the reverse thread hip lag screw 400 of FIG. 4, a reverse thread Smith+Nephew Trigen Intertan lntertrochanteric Antegrade Nail system, and the like. The head 530 includes a uniquely-configured coupling mechanism 537, which here is shown as a ¾ circle-shaped coupling mechanism 537 and would require a reciprocally-shaped driving mechanism (not shown) to be able to mate with and turn the uniquely-configured coupling mechanism 537.

FIG. 6 is a plan view of an end of a head of a reverse thread-type screw with a uniquely-configured drive coupling, in accordance with another embodiment of the disclosed subject matter. In FIG. 6, a head 630 of a reverse thread-type screw 600, which can include, for example, but is not limited to, the reverse thread screw 300 of FIG. 3, the reverse thread hip lag screw 400 of FIG. 4, a reverse thread Smith+Nephew Trigen Intertan lntertrochanteric Antegrade Nail system, and the like. The head 630 includes a uniquely-configured coupling mechanism 637, which here is shown as an elongated hexagonal-shaped coupling mechanism 637 and would require a reciprocally-shaped driving mechanism (not shown) to be able to mate with and turn the uniquely-configured coupling mechanism 637.

Alternatively, in other embodiments, a uniquely-configured coupling mechanism could be included that has at least two different depth levels and each level has a different configuration to prevent the screw from being advanced or removed by a reciprocally-shaped driving mechanism unless it is fully inserted into the coupling mechanism with differently configured depth levels. The above-described two shapes in FIGS. 5 and 6, as well as the possible multiple depth levels, are merely exemplary of the possible shapes of the uniquely-configured coupling mechanism and numerous other shapes and configurations are contemplated.

In the various configurations and embodiments, a reverse thread tap accompanies the reverse thread bone screw where the helix angle slopes upward to the left such that counter clockwise rotation would drive forward axial advancement and the dimensions and thread pitch of the reverse thread tap are the same as a corresponding traditional thread tap.

A reverse thread hip lag screw is an example embodiment of an application for a reverse thread bone screw. For example, a reverse thread hip lag screw for use in sliding hip screw and cephalomedullary nail constructs for osteosynthesis of select hip fractures. For fixation of left sided hip fractures, a reverse thread hip lag screw, where counter clockwise rotation would drive forward axial advancement, would produce the more favorable flexion force at the fracture (as occurs during traditional thread lag screw insertion for right sided hip fracture fixation), thus preventing the fixation implant from contributing to poor quality reduction with expected improvement in fracture healing and clinical outcomes. This premise applies to hip lag screws used in both sliding hip screw constructs and cephalomedullary nail constructs. This premise can be extended to dual lag screw cephalomedullary nails, commonly called “reconstruction nails” or “recon nails”, as well as cannulated screw systems used to treat some femoral neck fracture patterns.

Alternative configurations include reverse thread hip lag screws for sliding hip screw constructs; reverse thread hip lag screws for cephalomedullary nail constructs; reverse thread hip lag screws for dual lag screw cephalomedullary nail constructs; reverse thread fully threaded and partially threaded cannulated screws. The embodiments of the invention may be readily applied other anatomic locations and/or fracture patterns where conversion of a clockwise rotational force to a counter clockwise rotational force is beneficial.

In some instances, careful intraoperative monitoring with fluoroscopy, and is some cases direct fracture visualization, may be preferred during implant insertion for hip fracture fixation to ensure that reduction is maintained.

It will be apparent to those skilled in the art that various modifications and variations can be made in the reverse thread bone screw of the present invention without departing from the spirit or scope of the invention. Thus, it is intended that the present invention cover the modifications and variations of this invention provided they come within the scope of the appended claims and their equivalents.

What is claimed is:

1. A reverse thread bone screw comprising:
a substantially cylindrical body portion having a longitudinal axis with reverse angle threads formed around a distal end of the body portion, and a unique drive coupling formed in a proximal end of the body portion; and
a predefined radiographic identifier adhered to or embedded in the body portion.

2. The reverse thread screw of claim 1, wherein the reverse thread bone screw is a reverse thread screw hip lag screw.

3. The reverse thread hip lag screw of claim 2, wherein the unique driving mechanism is configured to accept a unique, reciprocally-shaped driving tool.

4. The reverse thread hip lag screw of claim 2, wherein the unique, reciprocally-shaped driving tool is configured to drive and remove the reverse thread hip screw in directions opposite to a non-reverse thread hip screw.

5. The reverse thread hip lag screw of claim 4, wherein the unique driving mechanism formed in a proximal end of the body portion is not compatible with standard screw driving tools for non-reverse threaded screws.

6. The reverse thread hip lag screw of claim 2, wherein the predefined radiographic identifier comprises:
a carbide ring connected at a base of the reverse angle threads; or
one or more beads of increased radiographic density connected to the body portion.

7. The reverse thread hip lag screw of claim 2, wherein the reverse angle threads are formed around a fixed length of the distal end of the body portion.

8. The reverse thread hip lag screw of claim 7, wherein the fixed length of the distal end of the body portion is less than 1 inch.

9. The reverse thread hip lag screw of claim 7, wherein the fixed length of the distal end of the body portion is less than ½ inch.

10. An reverse thread screw comprising:
a substantially cylindrical body portion having a longitudinal axis with reverse angle threads formed around a distal end of the body portion and the reverse angle threads extending the length of the body portion to terminate adjacent to a proximal end of the body portion, and a unique drive coupling formed in the proximal end of the body portion; and
a predefined radiographic identifier adhered to or embedded in the body portion.

11. The reverse thread screw of claim 10, wherein the unique drive coupling is configured to accept a unique, reciprocally-shaped driving tool.

12. The reverse thread screw of claim 10, wherein the unique, reciprocally-shaped drive tool is configured to drive and remove the reverse thread hip screw in directions opposite to a non-reverse thread hip screw.

13. The reverse thread screw of claim 10, wherein the unique drive coupling formed in a proximal end of the body portion is not compatible with standard screw driving tools for non-reverse threaded screws.

14. The reverse thread screw of claim 10, wherein the predefined radiographic identifier comprises:
a carbide ring connected circumferentially around and to the body portion; or
one or more beads of increased radiographic density connected to the body portion.

15. The reverse thread screw of claim 14, wherein the carbide ring is connected at a base of the reverse angle threads.

16. The reverse thread screw of claim 14, wherein the one or more beads of increased radiographic density are arranged longitudinally along the body portion.

17. The reverse thread screw of claim 14, wherein the one or more beads of increased radiographic density are arranged circumferentially around the body portion.

18. An reverse thread screw kit comprising:
a reverse thread screw comprising:
a substantially cylindrical body portion having a longitudinal axis with reverse angle threads formed around a distal end of the body portion and the reverse angle threads extending the length of the body portion to terminate adjacent to a proximal end of the body portion, and a unique drive coupling formed in the proximal end of the body portion; and
a predefined radiographic identifier adhered to or embedded in the body portion;
a reverse thread tap configured to provide reciprocally configured threads to engage the threads of the reverse thread screw; and
a reverse thread drive mechanism reciprocally configured to engage the unique drive coupling of the reverse thread screw.
